(19) United States
(12) Patent Application Publication
(10) Pub. No.: US 2023/0030847 A1
(43) Pub. Date: Feb. 2, 2023

(54) ANTI-MICROBIAL SULFONAMIDE PHARMACEUTICAL COMPOSITIONS FOR EXTERNAL USE

(71) Applicant: Rem Brands, Inc., Walton, KY (US)

(72) Inventors: David J. Schneider, Union, KY (US); Jonathan Schneider, Walton, KY (US); Joshua D. Schneider, Union, KY (US)

(21) Appl. No.: 17/877,361

(22) Filed: Jul. 29, 2022

Related U.S. Application Data
(60) Provisional application No. 63/226,998, filed on Jul. 29, 2021.

(51) Int. Cl. A61K 31/18; A61K 9/107; A61K 9/06; A61K 9/00; A61P 31/02; A61P 19/00

(52) U.S. Cl. CPC A61K 31/18; A61K 9/107; A61K 9/06; A61K 9/0073; A61P 31/02; A61P 19/00

(57) ABSTRACT

Disclosed herein are compositions useful for the treatment and/or prevention of various skin conditions, including infections, via external and/or topical administration to a patient. The compositions comprise an effective amount of a halo active aromatic sulfonamide compound having the structure of Formula (I):

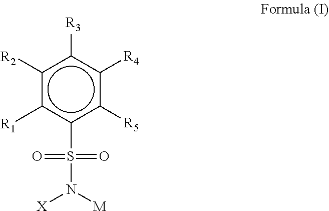

wherein R₁, R₂, R₃, R₄, and R₅, X, and M are defined herein. The compositions described may be used to treat and/or prevent a variety of skin conditions, including acne, eczema, rosacea, hives, psoriasis, athlete's foot, candidiasis, shingles, chicken pox, cold sores, bacterial infections, viral infections, fungal infections, and the like. The compositions can be externally and/or topically administered to a patient, for example, as a spray or a wipe. Further, the compositions can also be externally and/or topically administered to a patient by, for example, applying the composition to a medical device that is then implanted into the patient or contacted with the patient.

### CROSS-REFERENCE TO RELATED APPLICATIONS

This application claims priority to U.S. Provisional Patent Application Ser. No. 63/226,998, filed on Jul. 29, 2021, which is incorporated by reference in its entirety.

### BACKGROUND

The present disclosure relates to treating and/or preventing a variety of skin conditions, including infections, in a patient using a composition comprising a halo active aromatic sulfonamide compound. The methods and compositions disclosed herein find particular application in the treatment of patients by external and/or topical administration.

Infections occur when an organism gets access to inter-cellular and intra-cellular regions underneath the outer-protective membranes of the skin. Punctures, injections, bites, cuts, wounds, surgery, splits between skin and mucous membranes, organ or tissue transplants, among others, are all examples which could lead to infection. In particular, surgical site infections (SSI) are one of the most common and costly healthcare-associated infection causes among hospitalized patients. Further, infections may occur when foreign objects (e.g. medical devices and instruments) are not properly sterilized prior to use. Infections, such as bacterial infections, viral infections, and fungal infections, can lead to serious and life-threatening conditions. If not addressed, infections can grow, spread, and lead to secondary complications.

Currently, the risk of infections in a healthcare setting are addressed using a variety of preparations for medical instruments and surgical sites (e.g. a patient's skin). Depending on the type, design, and intended use of the medical instrument, one or more physical and/or chemical processes may be used to sterilize the instrument prior to or between operations. For example, steam sterilization, dry heat sterilization, chemical sterilization using a gas like ethylene oxide, and radiation are commonly used. Similarly, depending on the location of the surgical site and nature of the operation, a combination of skin preparations may be used, including a chlorhexidine-based solution, an iodine-based solution, as well as the like with an alcohol component, for example.

However, outcomes can vary drastically, and insufficient treatments and/or skin preparation can have serious consequences. Not utilizing a suitable or sufficient sterilization method can lead to the transmission of infectious diseases and lead to life-threatening conditions. Additionally, insufficient methods may prevent a medical device or instrument from even being approved by regulatory bodies such as the Food and Drug Administration (FDA). Further, certain pathogens are becoming drug-resistant, and thus traditional pharmaceuticals may no longer be effective in treating such infections. The threat of, for example, antibiotic-resistant bacteria to the world's population is serious and growing. Thus, it would be desirable to provide compositions that could be applied externally, either to a medical instrument or device or to a patient's skin (e.g. a surgical site) in order to treat or prevent a wide range of skin conditions, including those which have become resistant to one or more traditional pharmaceuticals, so as to provide greater certainty of effective treatment.

### BRIEF DESCRIPTION

Disclosed herein are compositions useful for the treatment and/or prevention of infections that affect the external pathophysiology of a patient. In some particular embodiments, these compositions are administered by topical administration to a patient. The compositions comprise an effective amount of a halo active aromatic sulfonamide compound having the structure of Formula (I):

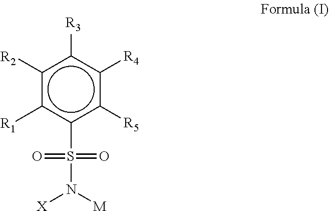

wherein R₁, R₂, R₃, R₄, and R₅, X, and M are defined herein.

The compositions described may be used to treat and/or prevent a variety of infections, including bacterial infections, viral infections, fungal infections, and the like. The compositions can be topically administered to a patient, for example, as a spray or a wipe. Further, the compositions can also be topically administered to a patient by, for example, applying the composition to a medical device that is then implanted into the patient or contacted with the patient. In some embodiments, the compositions can be taken orally, intrathecally, nasally, opthalmically, rectally, topically, enterally, transdermally, buccally, vaginally, intraurethrally, subcutaneously, sublingually, intravenously, inter-dermally, epidurally, intraperiotoneally, or intramuscularly to treat the external pathophysiology.

In accordance with one aspect of the present disclosure, a composition for administration to a patient is provided that is effective for the treatment and/or prevention of an external infection and/or other skin condition in the patient. The composition comprises at least a halo active aromatic sulfonamide compound. The halo active aromatic sulfonamide compound may have a structure according to Formula (I) as described above. In some embodiments, the halo active aromatic sulfonamide compound may have a structure according to Formula (II):

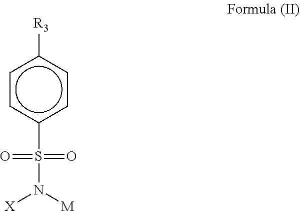

wherein R₃ is COOR′;
R′ is hydrogen, an alkali metal, an alkaline earth metal, substituted C₁-C₁₂ alkyl, unsubstituted C₁-C₁₂ alkyl, substituted aromatic, or unsubstituted aromatic;
X is halogen; and
M is an alkali or alkaline earth metal.

In certain embodiments, the compositions may comprise a halo active aromatic sulfonamide compound having a structure according to Formula (III):

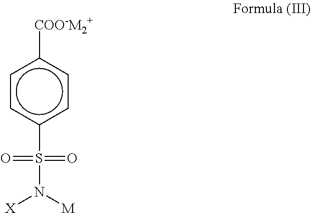

wherein M₂ is hydrogen, an alkali metal, or an alkali earth metal;
X is halogen; and
M is independently an alkali or alkaline earth metal.

In accordance with a further aspect of the present disclosure, a delivery device is provided for administering a composition comprising a halo active aromatic sulfonamide compound. The delivery device may be suitable and/or adapted for external administration of the composition.

In accordance with an additional aspect of the present disclosure, provided are methods for treating a patient suffering from an infection or is at risk of developing an infection using a composition. The methods include at least the step of externally administering a pharmaceutically effective amount of the composition to the patient. In some embodiments, the composition may be externally administered by contacting the patient's skin with the composition. In other embodiments, the composition may be externally administered by contacting the composition to a medical instrument or device that is to be contacted with the patient and/or implanted into the patient. The composition comprises a halo active aromatic sulfonamide compound of Formula (I):

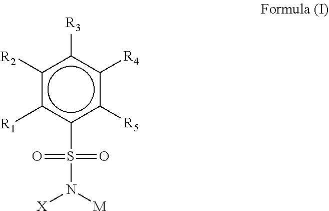

wherein R₁, R₂, R₃, R₄, and R₅ are independently selected from hydrogen, COOR′, CON(R″)₂, alkoxy, CN, NO₂, SO₃R″, halogen, substituted or unsubstituted phenyl, sulfonamide, halosulfonamide, N(R″)₂, substituted or unsubstituted C₁-C₁₂ alkyl, and substituted or unsubstituted aromatic;
R′ is hydrogen, an alkali metal, an alkaline earth metal, substituted C₁-C₁₂ alkyl, or unsubstituted C₁-C₁₂ alkyl; and
R″ is hydrogen or substituted or unsubstituted C₁-C₁₂ alkyl, where the two R″ groups in CON(R″)₂ and N(R″)₂ may be independently selected;
X is halogen; and
M is an alkali or alkaline earth metal.

These and other non-limiting features or characteristics of the present disclosure will be further described below.

### DETAILED DESCRIPTION

Halo active aromatic sulfonamide organic compounds have been known in general to reduce or eliminate odor. Chloramine-T is an example of a sulfonamide organic compound which has been used in many applications. The usefulness of chloramine-T is predicated on its ability to release an active chloride ion when needed on demand, immediately after which it simultaneously generates an active aromatic sulfo nitrene companion ion. The chlorine atom has a +1 formal charge in a hypochlorite ion, ClO⁻, which is the form taken by the chlorine atom when dissociated from the sulfonamide compound. Reference to the chlorine atom as having a +1 or 1⁻ charge may be used in this application interchangeably because this terminology has no effect on the compound itself or its use.

It has been found that halo active aromatic sulfonamide organic compounds can also have an antimicrobial effect that can extend over long periods of time when applied to surfaces in places such as a hospital, nursing home or long-term care facility, school, jail or prison, a vehicle, a house, a gym or workout facility, or a supermarket. Compared with typical disinfectants such as bleach, hydrogen peroxide, and peracetic acid, hydrates of halo active aromatic sulfonamide organic compounds continue to exhibit disinfectant ability over long time periods, such as over 24 hours, over 48 hours, over 72 hours, or even as long as 168 hours. Furthermore, such disinfectants are generally caustic to bodily tissues.

It has now been found that halo active aromatic sulfonamide organic compounds and compositions containing such compounds can obtain surprisingly effective antimicrobial results when administered to a patient in order to treat or prevent infections that affect the external pathophysiology of the patient. For example, it is contemplated that the compositions disclosed herein could be used to treat a variety of other skin conditions, including but not limited to, acne, eczema, rosacea, hives, psoriasis, athlete's foot, candidiasis, shingles, chicken pox, cold sores, and the like. Furthermore, it has been found that such compositions and administrations provide acceptable caustic effect to body tissues of humans, mammals, and other animals, thereby allowing for effective pharmaceutical application.

Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function.

### Definitions

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 to 10” is inclusive of the endpoints, 2 and 10, and all the intermediate values).

The term “about” can be used to include any numerical value that can vary without changing the basic function of that value. When used with a range, “about” also discloses the range defined by the absolute values of the two endpoints, e.g. “about 2 to about 4” also discloses the range “from 2 to 4.” The term “about” may refer to plus or minus 10% of the indicated number.

The term “ambient temperature” refers to a temperature of 20° C. to 25° C.

Compounds are described using standard nomenclature. For example, any position not substituted by any indicated group is understood to have its valency filled by a bond as indicated, or a hydrogen atom. A dash (“-”) that is not between two letters or symbols is used to indicate a point of attachment for a substituent. For example, the aldehyde group —CHO is attached through the carbon of the carbonyl group.

The term “alkyl” refers to a radical composed entirely of carbon atoms and hydrogen atoms which is fully saturated. The alkyl radical may be linear, branched, or cyclic, and such radicals may be referred to as linear alkyl, branched alkyl, or cycloalkyl.

The term “aromatic” refers to a radical that has a ring system containing a delocalized conjugated pi system with a number of pi-electrons that obeys HOckel's Rule. The ring system may include heteroatoms (e.g. N, S, Se, Si, 0), or may be composed exclusively of carbon and hydrogen. Exemplary aromatic groups include phenyl, thienyl, naphthyl, and biphenyl. The term “aromatic”, as used herein, does not refer to a smell detected by the nose.

The term “aryl” refers to an aromatic radical composed exclusively of carbon and hydrogen. Exemplary aryl groups include phenyl, naphthyl, and biphenyl.

The term “heteroaryl” refers to an aromatic radical containing at least one heteroatom. Exemplary heteroaryl groups include thienyl. Note that “heteroaryl” is a subset of “aromatic”, and is exclusive of “aryl”.

The term “alkoxy” refers to an alkyl radical which is attached to an oxygen atom, i.e. —O—CₙH₂ₙ₊₁, to a molecule containing such a radical.

The term “halogen” refers to fluorine, chlorine, bromine, and iodine.

The term “substituted” refers to at least one hydrogen atom on the named radical being substituted with another functional group, such as halogen, —CN, or —NO₂. Besides the aforementioned functional groups, an aromatic group may also be substituted with alkyl or alkoxy. An exemplary substituted aryl group is methylphenyl.

The term “alkali metal” refers to lithium, sodium, and potassium.

The term “alkaline earth metal” refers to magnesium and calcium.

The term “body” means the body of humans and/or animals; the term “subject” and “patient” means the body of such a human and/or animal.

As used herein, when referring to a “microbial,” “microbe,” or “pathogen,” the term may refer to any of a virus, a bacteria, a spore, a fungi, or the like, which may cause an infection in a human and/or animal.

Unless otherwise indicated, the term “infection” is intended to encompass any type of infection, such as a bacterial infection, viral infection, fungal infection, and the like.

As used herein, when referring to “external administration” of a composition, the term refers to the administration of the composition outside of the subject's body, as opposed to inside the subject (e.g. injection).

The terms “treatment” and “treating” are intended to encompass also prophylaxis, therapy, and cure. Accordingly, in one aspect, a treatment involves preventing or delaying or slowing the onset of a condition, disease, or disorder (e.g. the symptoms associated with the disease, condition, or disorder). In another aspect, a treatment involves treating (e.g. minimizing or reducing or slowing the development or reversing) an existing condition, disease, or disorder (e.g. the symptoms associated with the disease, condition, or disorder). In one embodiment, a treatment provides a cure for a condition, disease, or disorder (e.g. eliminates the condition, disease, or disorder).

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject copolymer and/or composition from one organ, or portion of the body, to another organ, or portion of the body. Each carrier must be “acceptable” in the sense of being compatible with the other ingredients of the formulation and not unduly injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

The term “pharmaceutically effective amount”, as defined herein, refers to an amount of the sulfonamide that is capable of providing therapeutic effect to the patient when used. This amount may be measured in absolute amounts (e.g. grams) or in relative amounts (e.g. mg/kg).

Compositions

Disclosed in various embodiments herein are compositions for administration in a patient and effective for the treatment or prevention of an external infection or other skin condition in the patient. The compositions of the present disclosure comprise (A) a halo active aromatic sulfonamide compound, as described further herein. The compositions can also include additional components, including a buffering agent (B), a surfactant (C), and a solvent (D), as well as others, and any combination of these additional ingredients.

Halo Active Aromatic Sulfonamide Compound

The halo active aromatic sulfonamide compounds (A) disclosed herein are stable and do not decompose in aqueous solution, allowing the composition to have a long shelf life and persistent antimicrobial effect. The halo active aromatic sulfonamide compounds are also very soluble in water, low in toxicity, and have minimal bleach odor.

The halo active aromatic sulfonamide compounds (A) disclosed herein are stable and do not decompose in aqueous solution, allowing the composition to have a long shelf life and persistent therapeutic effect. The halo active aromatic sulfonamide compounds are also very soluble in water, low in toxicity, and have minimal bleach odor.

In general, the halo active aromatic sulfonamide compound is present in the composition in the amount of about 0.0001 wt % to about 100 wt %, including from about 0.0001 wt % to about 0.001 wt %, from about 0.001 wt % to about 0.01 wt %, from about 0.01 wt % to about 0.1 wt %, from about 0.1 wt % to about 1 wt %, from about 1 wt % to about 5 wt %, from about 5 wt % to about 10 wt %, from about 10 wt % to about 15 wt %, from about 15 wt % to about 20 wt %, from about 20 wt % to about 25 wt %, from about 25 wt % to about 30 wt %, from about 30 wt % to about 35 wt %, from about 35 wt % to about 40 wt %, from about 40 wt % to about 45 wt %, from about 45 wt % to about 50 wt %, from about 50 wt % to about 55 wt %, from about 55 wt % to about 60 wt %, from about 60 wt % to about 65 wt %, from about 65 wt % to about 70 wt %, from about 70 wt % to about 75 wt %, from about 80 wt %, from about 80 wt % to about 85 wt %, from about 85 wt % to about 90 wt %, from about 90 wt % to about 95 wt %, and from about 95 wt % to about 100 wt %, including any combination of endpoints thereof. In particular embodiments, the composition may consist essentially of the halo active aromatic sulfonamide compound. In further embodiments, the halo active aromatic sulfonamide compound is present in the composition in the amount of about 0.0001 wt % to about 45 wt %, including from about 0.0001 wt % to about 30 wt %, from about 0.0001 wt % to about 25 wt %, from about 0.0001 wt % to about 20 wt %, from about 0.1 wt % to about 20 wt %, from about 0.0001 wt % to about 5 wt %, from about 1 wt % to about 2 wt %, from about 2 wt % to about 3 wt %, from about 3 wt % to about 4 wt %, and from about 4 wt % to about 5 wt %. In particular embodiments, the composition may consist essentially of the halo active aromatic sulfonamide compound.

In some embodiments, the composition comprises from about 0.021 wt % to about 0.085 wt % of the halo active aromatic sulfonamide compound, or from about 0.085 wt % to about 0.17 wt % of the halo active aromatic sulfonamide compound. In other embodiments, the composition may comprise from about 210 milligrams (mg) to about 850 milligrams of the halo active aromatic sulfonamide compound per liter of composition, or from about 850 milligrams to about 1700 milligrams of the halo active aromatic sulfonamide compound per liter of the composition. In some embodiments, the dosage of the composition is such that the total amount of the halo active aromatic sulfonamide compound administered to the patient is at least 100 milligrams per kilogram (kg) of patient weight, or at most 460 milligrams per kilogram of patient weight. In still further embodiments, the composition may comprise at least 0.021 wt % of the halo active aromatic sulfonamide compound, or at least 0.085 wt % of the halo active aromatic sulfonamide compound, or at least 0.17 wt % of the halo active aromatic sulfonamide compound, or at least 210 milligrams of the halo active aromatic sulfonamide compound per liter of the composition, or at least 850 milligrams of the halo active aromatic sulfonamide compound per liter of the composition, or at least 1700 milligrams of the halo active aromatic sulfonamide compound per liter of the composition.

The halo active aromatic sulfonamide compound used in the compositions of the present disclosure can have the structure of base Formula (I):

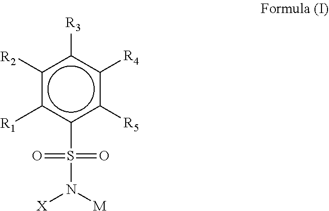

wherein R₁, R₂, R₃, R₄, and R₅ are independently selected from hydrogen, COOR′, CON(R″)₂, alkoxy, CN, NO₂, SO₃R″, halogen, substituted or unsubstituted phenyl, sulfonamide, halosulfonamide, N(R″)₂, substituted or unsubstituted C₁-C₁₂ alkyl, and substituted or unsubstituted aromatic;
R′ is hydrogen, an alkali metal, an alkaline earth metal, substituted C₁-C₁₂ alkyl, or unsubstituted C₁-C₁₂ alkyl; and
R″ is hydrogen or substituted or unsubstituted C₁-C₁₂ alkyl, where the two R″ groups in CON(R″)₂ and N(R″)₂ may be independently selected;
X is halogen; and
M is an alkali or alkaline earth metal.

Generally, M is sodium or potassium. X is generally chlorine, bromine, fluorine, or iodine, and in particular embodiments is chlorine. Compounds of Formula (I) may or may not be hydrated. In particular embodiments, the compounds of Formula (I) are a trihydrate (i.e., n=3) or a hexahydrate (i.e. n=6). In other embodiments, the compound is in a solid form, such as a powder.

When the phenyl and/or alkyl group is substituted, one or more hydrogen atoms may be independently replaced with hydroxyl or halogen.

In particular embodiments of Formula (I), R₃ is methyl, COOH, or COOM₁; R₁, R₂, R₄, and R₅ are independently selected from hydrogen, COOH, COOM₁, COOR′, CON(R″)₂, alkoxy, CN, NO₂, SO₃R″, halogen, substituted or unsubstituted phenyl, sulfonamide, halosulfonamide, N(R″)₂, substituted or unsubstituted C₁-C₁₂ alkyl, and substituted or unsubstituted aromatic; X is halogen; and M₁ is an alkali or alkaline earth metal.

In further embodiments, R₃ is methyl, COOH, or COOM₁; R₁, R₂, R₄, and R₅ are independently selected from hydrogen, COOH, COOM₁, COOR′, CON(R″)₂, alkoxy, CN, NO₂, SO₃R″, halogen, substituted or unsubstituted phenyl, sulfonamide, halosulfonamide, N(R″)₂, substituted or unsubstituted C₁-C₁₂ alkyl, and substituted or unsubstituted aromatic; X is halogen; M is an alkali or alkaline earth metal; and at least one of R₁, R₂, R₄, and R₅ is not hydrogen.

In yet other embodiments of Formula (I), R₃ is selected from COOH, COOM₁, COOR′, CON(R″)₂, CN, NO₂, halogen, and substituted or unsubstituted C₂-C₁₂ alkyl; R₁, R₂, R₄, and R₅ are independently selected from hydrogen, COOH, COOM₁, COOR′, CON(R″)₂, alkoxy, CN, NO₂, SO₃R″, halogen, substituted or unsubstituted phenyl, sulfonamide, halosulfonamide, N(R″)₂, substituted or unsubstituted C₁-C₁₂ alkyl, and substituted or unsubstituted aromatic; X is halogen; and M is an alkali or alkaline earth metal.

In still other embodiments of Formula (I), R₁, R₂, R₃, R₄, and R₅ are independently selected from hydrogen, COOH, COOM₁, NO₂, halogen, N(R″)₂, substituted or unsubstituted C₁-C₁₂ alkyl, and substituted or unsubstituted aromatic; X is halogen; and M is an alkali or alkaline earth metal.

In yet other embodiments of Formula (I), R₂ and R₄ are identical to each other; and R₁, R₃, and R₅ are hydrogen.

In yet other embodiments of Formula (I), R₂ and R₄ are hydrogen; and R₁, R₃, and R₅ are identical to each other.

In more specific embodiments of Formula (I), R₃ is selected from COOH, COOM₁, COOR′, and CON(R″)₂. Most desirably, R₃ is COOH or COOM₁, while R₁, R₂, R₄, and R₅ are hydrogen.

In other embodiments of Formula (I), R₁, R₂, R₃, R₄, and R₅ are independently selected from hydrogen, COOH, COOM₁, COOR′, CON(R″)₂, NO₂, halogen, N(R″)₂, substituted or unsubstituted C₁-C₁₂ alkyl, and substituted or unsubstituted aromatic; wherein at least one of R₁, R₂, R₃, R₄, and R₅ is not hydrogen; X is halogen; and M is an alkali or alkaline earth metal.

In still other embodiments of Formula (I), R₃ is COOH or COOM₁; R₁, R₂, R₄, and R₅ are independently selected from hydrogen, NO₂, halogen, N(R″)₂, substituted or unsubstituted C₁-C₁₂ alkyl, and substituted or unsubstituted aromatic; X is halogen; and M is an alkali or alkaline earth metal. In further specific embodiments, at least one of R₁, R₂, R₄, and R₅ is not hydrogen.

In some embodiments of Formula (I), at least one of R₁, R₂, R₃, R₄, or R₅ are not hydrogen. In more specific embodiments of Formula (I), at least two of R₁, R₂, R₃, R₄, or R₅ are not hydrogen. In other words, the benzene ring contains the sulfonamide substituent and an additional one or two other substituents.

In other embodiments of Formula (I), the halo active aromatic sulfonamide compound has the structure of Formula (II):

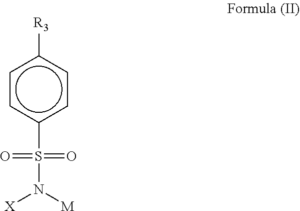

wherein R₃ is COOR′; R′ is hydrogen, an alkali metal, an alkaline earth metal, substituted C₁-C₁₂ alkyl, unsubstituted C₁-C₁₂ alkyl, substituted aromatic, or unsubstituted aromatic; X is halogen; and M is an alkali or alkaline earth metal. The N-chloro-4-carboxybenzenesulfonamide compound of Formula (II) is also referred to herein as BENZ. BENZ exhibits a lower chlorine smell than chloramine-T or chloramine-B. BENZ is also known as Monalazone Disodium, CAS #61477-95-0.

One particular sulfonamide compound contemplated for use is N-chloro-4-carboxybenzenesulfonamide (i.e. BENZ). This compound is shown below as Formula (III):

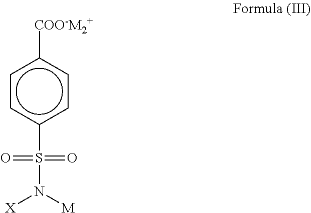

wherein M₂ is hydrogen, an alkali metal, or an alkali earth metal; X is halogen; and M is independently an alkali or alkaline earth metal. Desirably, M₂ is hydrogen, sodium, or potassium.

In other embodiments, one or more of R₁, R₂, R₃, R₄, and R₅ are substituted with —COOR′ (and the others are hydrogen). In this regard, it is believed that when the halo active aromatic sulfonamide compound has two or more ionic charges, that the compound has higher antimicrobial performance.

In further embodiments, the composition may comprise a halo active aromatic sulfonamide compound that is a disodium salt. That is, the halo active aromatic sulfonamide compound may have the general structure of Formula (I) wherein the functional groups R₁, R₂, R₃, R₄, and R₅ contain two sodium atoms that dissociate upon hydration.

In still further embodiments, the composition may comprise a halo active aromatic sulfonamide compound that is a trisodium salt. That is, the halo active aromatic sulfonamide compound may have the general structure of Formula (I) wherein the functional groups R₁, R₂, R₃, R₄, and R₅ contain three sodium atoms that dissociate upon hydration.

In some embodiments, the composition may comprise a halo active aromatic sulfonamide compound that is a dicarboxy sulfonamide. That is, the halo active aromatic sulfonamide compound may have the general structure of Formula (I) wherein two of the functional groups R₁, R₂, R₃, R₄, and R₅ are —COOH.

In particular embodiments, the composition may be formulated for improved fat solubility. That is, in certain embodiments, the composition comprises a halo active aromatic sulfonamide compound wherein at least one of R₁, R₂, R₃, R₄, or R₅ is a long chain aliphatic group, such as a substituted or unsubstituted C₁-C₁₂ alkyl.

It is also believed that the compounds of Formula (I) do not have mutagenic properties, which may reduce the evolution of resistance to these compounds. Alternatively, their use might lower the total microbial load which is encountered by antibiotics used in conjunction therewith.

One significant difference between the compounds of Formula (I) and conventional sulfa drugs is the presence of a stable halogen on the nitrogen atom of the sulfonamide group, which provides a higher efficacy per mole than any sulfa drug. This results in much quicker kill without the side effects of typical halogen chemistry. In addition, the sulfonamide compound selectively kills bacteria, viruses, and fungi.

One of ordinary skill in the art would not have expected such sulfonamide compounds to be effective for external administration or as a topical formulation for use as a pharmaceutical product because prior uses of such compounds were almost always labeled with a warning not to use it on your skin or body.

Additional Components

The compositions of the present disclosure may include or exclude one or more additional components, including, for example, at least one of a buffering agent (B), a surfactant (C), and a solvent (D), among others.

A buffering agent (B) can be included to maintain the composition within a desired pH range. In particular embodiments, the composition may have a pH of from about 6 to about 10. In specific embodiments, the composition may have a pH of about 6, or about 6.5, or about 7, or about 7.5, or about 8, or about 8.5, or about 9, or about 9.5, or about 10. The buffering agent can be added up to the limit of solubility in the compositions. In particular embodiments, the preferred weight ratio of the sulfonamide compound to the buffering agent is from about 50:1 to about 1:1, or from about 50:1 to about 2:1, or from about 20:1 to about 2:1.

Exemplary buffering agents include sodium bicarbonate, potassium bicarbonate, sodium carbonate, potassium carbonate, acetate buffers (such as sodium acetate), phosphate buffers (such as tri and di sodium phosphate and mixtures thereof, pH blended phosphates, sulfate buffers (such as di and tri sodium sulfate), and mixtures thereof. The preferred buffering agent is sodium bicarbonate.

A surfactant (C), or wetting agent, can also be added to the composition. The surfactant decreases surface tension, allowing the sulfonamide compound to be move more freely upon administration. Both non-ionic and anionic surfactants can be used. However, in some specific embodiments, a surfactant is not used. The surfactant can be present in the composition in the amount of about 0.0001 wt % to about 5 wt %, including from about 0.0001 wt % to about 0.001 wt %, from about 0.001 wt % to about 0.01 wt %, from about 0.01 wt % to about 0.1 wt %, from about 0.1 wt % to about 1 wt %, from about 1 wt % to about 2 wt %, from about 2 wt % to about 3 wt %, from about 3 wt % to about 4 wt %, and from about 4 wt % to about 5 wt %.

Additionally, a solvent (D), which acts as a carrier vehicle for externally administering the halo active aromatic sulfonamide compound to a patient, may be included. In particular embodiments, the solvent can make up the majority of the composition. That is, the compositions may comprise a balance amount of the solvent (i.e. comprise the remaining portion of the composition). In specific embodiments, the solvent may be water, a pharmaceutically-acceptable carrier, or a combination thereof.

The composition may also include excipients and adjuvants as desired. Particular excipients include buffering agents, surfactants, preservative agents, bulking agents, polymers, and stabilizers. Buffering agents are used to control the pH of the composition. Surfactants are used to stabilize proteins, inhibit protein aggregation, inhibit protein adsorption to surfaces, and assist in protein refolding. Exemplary surfactants include Tween 80, Tween 20, Brij 35, Triton X-10, Pluronic F127, and sodium dodecyl sulfate. Preservatives are used to prevent microbial growth. Examples of preservatives include benzyl alcohol, m-cresol, and phenol. Bulking agents are used during lyophilization to add bulk. Hydrophilic polymers such as dextran, hydroxyl ethyl starch, polyethylene glycols, and gelatin can be used to stabilize proteins. Polymers with nonpolar moieties such as polyethylene glycol can also be used as surfactants. Protein stabilizers can include polyols, sugars, amino acids, amines, and salts. Suitable sugars include sucrose and trehalose. Amino acids include histidine, arginine, glycine, methionine, proline, lysine, glutamic acid, and mixtures thereof. Proteins like human serum albumin can also competitively adsorb to surfaces and reduce aggregation. It should be noted that particular compounds can serve multiple purposes. For example, histidine can act as a buffering agent and an antioxidant. Glycine can be used as a buffering agent and as a bulking agent. Adjuvants may be used to improve immune response. Examples of adjuvants may include aluminum salts, AS04, MF59, AS01 B, and CpG 1018.

The dosage form of the composition may vary. For example, the dosage form may be an aerosol, a cream, an emulsion, a gel, a lotion, an ointment, a paste, a powder, a soap, a spray, a suspension, or a tape. These dosage forms are especially suitable for external administration, such as upon the skin of the patient or user.

In an aerosol, the composition is packaged under pressure and also contains a propellant. When actuated, a dose of the composition is delivered in the form of a plume or mist of particles/droplets, or in other words in dry or wet form. For example, the composition may be sprayed upon the skin. Examples of propellants can include nitrous oxide, nitrogen (N₂), carbon dioxide, HFA 134a, HFA 227, certain hydrocarbons like propane or butane, ethers, and combinations thereof. In contrast, a spray is not pressurized, and the plume or mist is generated by discharge through the nozzle.

An emulsion includes two immiscible liquid phases that are usually stabilized with one or more suitable emulsifying agents. Those two phases may include an aqueous phase and an organic phase, with the sulfonamide (A) being present in one of the phases. Examples of emulsions include creams, lotions, and ointments. A cream is semisolid, and typically contains more than 20 wt % water and less than 50 wt % of hydrocarbons, waxes, or polyols. A lotion is very similar to a cream, and is generally distinguished by being more fluid (i.e. a lower viscosity). An ointment is semisolid, and typically contains less than 20 wt % water and more than 50 wt % of hydrocarbons, waxes, or polyols. The organic phase of an emulsion can be formed from oils (mineral and/or vegetable), fatty alcohols, fatty acids, and/or fatty esters. Emulsifying agents can include surfactants, which can be non-ionic, cationic, anionic, or zwitterionic. A soap is essentially a solid emulsion.

A gel can be solid or semisolid, but is mostly liquid. A gel contains a gelling agent, such as starch, xanthan, guar gum, locust bean gum, gum karaya, gum tragacanth, gum Arabic, alginate, pectin, carrageenan, gelatin, gellan agar, methylcellulose, hydroxymethylcellulose, and/or carboxymethylcellulose. Other ingredients can include sugars, water, sweeteners, and flavoring agents. A paste is similar to a gel, and usually contains a high percentage of small, dispersed solids as well.

A powder is a solid (i.e. the sulfonamide (A)) in a finely divided state. A suspension typically consists of solid particles (i.e. the sulfonamide (A)) dispersed in a liquid phase.

A tape includes a substrate into which the sulfonamide (A) is impregnated. The substrate can be, for example, a woven or non-woven material. One side of the substrate is coated with an adhesive agent, which holds the tape in place without the need for additional material. A tape is also known as a bandage or a patch.

While not being limited by theory, it is believed the antimicrobial kill performance of the sulfonamide will extend over a long time period as well, so that new growths of microorganisms will also be eliminated. Extended treatment and prophylactic protection of patients is thus possible. Further, unlike other products such as bleach, which should not be used within a human or animal body, the present compositions may be externally administered to a patient because they surprisingly have minimal effects on healthy tissues.

The compositions can be externally or topically administered to a patient that is suffering from or is at risk of developing an infection, such as a bacterial infection, a viral infection, a fungal infection, and the like. In some embodiments, treatment using the disclosed compositions may be achieved by applying the composition to a medical device or a medical instrument outside of the patient's body, and the medical device and/or instrument is then contacted with the patient. The medical device may be, for example, a type of implant that is first treated with the composition and then implanted into the patient's body (i.e. the composition is applied to the external surface of the medical device). In other embodiments, treatment using the disclosed compositions may be achieved by applying the composition to a portion of the patient's skin. For example, prior to surgery, a surgical site on the patient's skin may be treated to prevent infection by covering or contacting the surgical site with the composition prior to surgery.

In accordance with certain aspects of the present disclosure, a delivery device is provided that can comprise the composition described above. The delivery device may be suitable and/or adapted for external or topically administration of the composition. In one embodiment, the delivery device may be a wipe containing the composition.

In some embodiments, it is contemplated that the composition containing the sulfonamide can be used as a hand sanitizer, a hand disinfectant, or a hand sterilant. To be a hand sanitizer, 99.9% of bacteria/viruses must be killed within one minute. To be a hand disinfectant, 99.999% of bacteria/viruses must be killed within five minutes. To be a hand sterilant, 99.9999% of bacteria/viruses/fungi/spores must be killed within five minutes. Typical hand sanitizers regulated by the FDA contain 60% to 95% (v/v) ethyl alcohol or 70% to 91.3% (v/v) isopropyl alcohol, along with small amounts of glycerol and hydrogen peroxide, and balance water. However, alcohol and hydrogen peroxide are only active when wet, and do not exist in a “dry” form. In addition, they only kill common and easy-to-kill germs. In contrast, the present compositions can provide extreme amounts of protection against hard-to-kill bacteria, viruses, fungi, and spores such as norovirus, Candida auris, and C. difficile.

Some conditions which affect the external pathophysiology can be treated by oral or injectable compositions. For example, oral antibiotics and oral antifungals are used to treat many skin conditions. It is thus contemplated that the compositions of the present disclosure may be provided for internal administration. Examples of some dosage forms which may be taken internally to affect the external pathophysiology include an aerosol, a capsule, an emulsion, a film, a gel, granules, a gum, an injection, a lozenge, a paste, pellets, a pill, a powder, a solution, a spray, a suppository, a suspension, or a tablet.

An aerosol/spray, emulsion, gel, paste, powder, and suspension have been previously described. The aerosol or spray may be inhaled through the mouth or the nose, or can be sprayed sublingually. Emulsions can be taken orally or parenterally, and can be injectable. Gels/pastes can be molded into a desired shape for oral delivery, and are colloquially called “gummies”. A powder can be taken orally. A suspension can ingested, injected, inhaled, or taken via ophthalmic and otic routes.

A capsule or pellet is a solid dosage form in which the composition is enclosed within a soluble container or shell. The shell may be a single piece, or may be made from two pieces. The shell can be hard or soft. The capsule can be made for immediate release of the composition, or can be a modified-release capsule. For example, the capsule may be coated with an enteric coating to protect the capsule from the gastric environment or may be modified as an extended-release capsule that releases the composition over an extended time period. The shell is made, for example from a gelatin or a cellulose polymer. Capsules and pellets are typically ingested.

A film is a thin sheet that can be taken orally, buccally, or sublingually. The film may contain one or more layers, and can be formulated with edible polymers or with water-soluble polymers. The dissolution rate of the film can be controlled.

Granules are a solid formed by agglomeration of smaller particles, and can also include excipients, binders, and/or solvents. They are typically taken orally.

A gum is a dosage form that is intended to be chewed rather than swallowed. They typically include a gum base, as well as plasticizers, softeners, and/or sugars.

An injection is typically a liquid form, such as a solution or suspension, which is intended to be injected into the body. A solution includes the sulfonamide (A) in a liquid form. A solution can be ingested, injected, or inhaled.

A lozenge is a solid form that is designed to dissolve or disintegrate slowly, and is typically taken orally. A lozenge typically is molded and includes gelatin, sugars, or other bases. A pill is a solid and is typically distinguished from capsules only by its manufacturing process. A pill is usually prepared from a wet mass which is molded. A pill is usually taken orally.

A suppository is intended to be taken rectally. The suppository typically includes a base such as cocoa butter gelatin, vegetable oils, or mixtures of various polymers such as polyethylene glycol.

A tablet is a solid typically made by compaction of powders or granules. Tablets can also be coated with an enteric coating or an extended-release coating. Tablets can be taken buccally, orally, chewed, or sublingually.

Methods

In accordance with still another aspect of the present disclosure, provided are methods for treating a patient with a composition, wherein the patient is suffering from a skin condition or is at risk of developing a skin condition. For example, the patient may be at risk of developing a skin infection, or may have already contracted a type of skin infection. In other embodiments, the patient may be suffering from or at risk of developing one or more of: acne; eczema; rosacea; hives; psoriasis; athlete's foot; candidiasis; shingles; chicken pox; cold sores. In particular embodiments, the skin condition may be a drug resistant skin infection. Further, the patient may be, for example, a human or an animal.

The methods disclosed can include at least the step of administering an amount of the composition to the patient. In particular embodiments, the composition may be externally administered by contacting the patient's skin with the amount of the composition. In further embodiments, the methods disclosed can include at least the step of providing an amount of the composition to a delivery device, and then externally administering the dosage of the composition to the patient via the delivery device (e.g. by contacting an external portion of the patient with the delivery device). In certain embodiments, the delivery device may be at least one of surgical gloves, gauze, wound dressing, and a skin graft, or the like. In other embodiments, the composition is internally administered, which treats the skin condition.

The composition comprises a halo active aromatic sulfonamide compound having the structure of Formula (I):

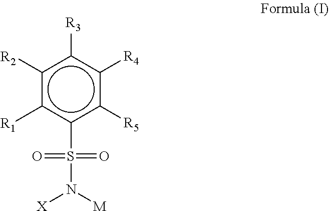

wherein R₁, R₂, R₃, R₄, and R₅ are independently selected from hydrogen, COOR′, CON(R″)₂, alkoxy, CN, NO₂, SO₃R″, halogen, substituted or unsubstituted phenyl, sulfonamide, halosulfonamide, N(R″)₂, substituted or unsubstituted C₁-C₁₂ alkyl, and substituted or unsubstituted aromatic;
R′ is hydrogen, an alkali metal, an alkaline earth metal, substituted C₁-C₁₂ alkyl, or unsubstituted C₁-C₁₂ alkyl; and
R″ is hydrogen or substituted or unsubstituted C₁-C₁₂ alkyl, where the two R″ groups in CON(R″)₂ and N(R″)₂ may be independently selected;
X is halogen; and
M is an alkali or alkaline earth metal.

An effective amount of composition may be administered to the patient and/or the medical device. In certain embodiments, the composition can comprise from about 0.0001 wt % to about 100 wt % of the halo active aromatic sulfonamide compound. In further embodiments, the compositions may include at least one of a buffering agent, a surfactant, and/or a solvent as described above. In alternative embodiments, the composition may exclude one or more of a buffering agent, a surfactant, and/or a solvent.

In particular embodiments, the composition may be incorporated into a delivery device suitable and/or adapted for external administration of the composition. For example, the composition may be formulated as a liquid, a foam, a gel, a semi-solid gel, a cream, a powder, or the like, and the delivery device may be a spray or a wipe. That is, the spray or wipe may be used to contact the portion of the patient's skin with the composition in a liquid form, a foam form, a gel form, a semi-solid gel form, a cream form, a powder form, or the like. Thus, in accordance with one aspect of the present disclosure, the methods of treating or preventing infection in a patient may include the step of spraying or wiping a portion of the patient's skin (e.g. the surgical site) with a wipe or solution containing the composition.

In further embodiments, the compositions can be externally administered to the medical device/instrument by, for example, contacting the device or instrument with the composition. In particular embodiments, the composition may be incorporated into a delivery device suitable and/or adapted for external administration of the composition. For example, the composition may be formulated as a solution. Thus, in accordance with another aspect of the present disclosure, the methods of treating or preventing infection in a patient may include the step of washing the medical device, instrument, implant, or the like, in a solution containing the composition.

The methods described herein may include externally administering the composition using a combination of formulations and/or delivery devices. For example, in some embodiments a spray may be used in a first step, and a wipe may be used in a second step.

In some embodiments, the dosage of the composition may be administered in one or more doses over a period of time. That is, the method may include the additional step of repeating the same or a different dosage of the composition after a period of time. For example, the method of treating a patient suffering from an infection and/or at risk of developing an infection can include administering a first dosage of the composition, waiting a first period of time, administering a second dosage of the composition, waiting a second period of time, and so forth, wherein the first and second doses of the composition may be the same or different. In other words, the dosage of the composition may be administered in two or more doses over a certain period of time (i.e., a treatment period). The first, second, and further waiting periods may each be from about 30 minutes to about 3 days. Further, the composition may be administered over an extended period of time. In particular embodiments, an effective dosage of the disclosed compositions may be administered about every one hour, or about every two hours, or about every four hours, or about every eight hours, or at least once per day, or at least twice per day, or about once per week, or about once per month. This administration regiment may be repeated a plurality of times over the duration of several hours to several months, or until the treatment and/or prevention of the skin condition is completed. Additionally, the method of administering the doses of the composition may vary from dose to dose (e.g. injection vs. inhalant). The composition can be externally administered or internally administered, as appropriate.

The compositions of the present disclosure are illustrated by the following non-limiting examples, it being understood that these examples are intended to be illustrative only and that the present application is not intended to be limited to the materials, conditions, process parameters and the like recited herein. All proportions are by weight unless otherwise indicated.

### EXAMPLES

### Example 1

Five rats (Rattus norvegicus) weighing 244 to 272 grams were gavaged with a single dose of liquid containing 20% N-chloro-4-carboxybenzenesulfonamide (BENZ) at a dosage of 5 g/kg body weight. After seven days, all five rats were still alive, and there was no evidence of toxicity.

Next, three rabbits were used for evaluation of primary skin irritation according to ISO 10993-10, Part 10. Two wet gauze patches (25 mm×25 mm) treated with 20% BENZ were applied to the skin of each rabbit in different locations. Each application site was abraded with four parallel epidermal abrasions prior to application of the treated gauze patch. The treated gauze was secured in place for 24 hours, then removed. The skin was then observed for erythema and edema at 1, 24, 48, and 72 hours after patch removal. There was no erythema or edema observed.

Next, 10 guinea pigs were used to test for delayed dermal contact sensitization according to ISO 10993-10, Part 10. A wet gauze patch (25 mm×25 mm) treated with 20% BENZ was applied to the intact skin of each guinea pick for six hours (±30 minutes), three times a week, over a three-week period for a total of nine applications. After a two-week recovery period, the gauze patch was applied again. The skin was then observed for evidence of dermal reaction at 24 and 48 hours after patch removal. No evidence was found of delayed dermal contact sensitization. This test was performed a total of three times with different guinea pigs, and the same results were obtained each time.

### Example 2

A bovine corneal opacity and permeability (BCOP) test was performed to determine the potential for ocular irritation.

Each dissected cornea was mounted in a holder that separated into anterior and posterior chambers. The two chambers were filled with a MEM solution, and an opacitometer was used to measure the opacity of the cornea. Next, the MEM solution was removed from the anterior chamber, and the epithelium of three bovine corneas were exposed to 0.75 mL of a 20% BENZ solution for 10 minutes in a manner that covered the entire cornea. The two chambers were then rinsed and refilled with fresh MEM solution. The corneas were then incubated at 32° C. for two hours, and the two chambers were again rinsed and refilled with fresh MEM solution. The opacity was measured again. The MEM solution was then removed from the anterior chamber and the anterior chamber was filled with 1.0 mL of 0.4% sodium fluorescein in Dulbecco's phosphate-buffered saline. After 90 minutes, the fluid in the posterior chamber was removed and the amount of dye that passed through the cornea was measured as the optical density at 490 nm by spectrophotometer. The In Vitro Irritancy Score (IVIS) was calculated to be −0.67, which is a perfect score.

### Example 3

Test swatches which had been impregnated with 2.5% BENZ were exposed to Staphylococcus aureus, Pseudomonas aeruginosa, and Aspergillus brasiliensis. A stack of two swatches was placed in a petri dish and then inoculated with 1.000 mL of an inoculum for one of the microorganisms. Following inoculation, the petri dishes were sealed and placed in a plastic bag/container, then incubated at optimal conditions for the given microorganism. Swatches were harvested at 0, 10 minutes, 30 minutes, 1 hour, and 6 hours after inoculation.

At harvesting, the swatch was transferred to a neutralization media, then vortex mixed for 1-2 minutes. The neutralization suspension was numbered and plated for 18-24 hours for bacteria and 24-72 hours for fungi at optimal incubation conditions.

As to S. aureus, 99.999% kill was obtained within 30 minutes.

As to P. aeruginosa, 99.999% kill was obtained within 10 minutes.

As to A. brasiliensis, 99.9% kill was obtained within 1 hour.

A similar test was run with Candida auris. A stack of four swatches was placed in a petri dish and then inoculated with 1.000 mL of the inoculum. Following inoculation, the petri dishes were sealed and placed in a plastic bag/container, then incubated at optimal conditions. 99.998% kill was obtained within 1 hour.

Next, a filter medium impregnated with 2.5% BENZ was exposed to a 0.200 mL volume of Rhinovirus 37, 151-1 strain, ATCC VR-1607 for a contact time of 30 minutes at 24.7-24.9° C. and 39.9-40.5% relative humidity.

After 30 minutes, the filter medium was transferred to a neutralization medium and vortexed for at least 15 seconds. A 0.1 mL aliquot was removed and a series of 10-fold dilutions was performed in 0.9 mL aliquots of test medium. Each dilution was inoculated into MRC-5 cells. Viral titer was then calculated. The TCID₅₀ represents the endpoint dilution where 50% of the cell cultures exhibited cytopathic effects, and was determined using the Spearman-Karber method. The BENZ filter medium demonstrated a >99.997% reduction in viral titer.

A similar experiment was performed with Influenza A virus, A/PR/8/34 strain, ATCC VR-1469. The BENZ filter medium demonstrated a >99.99997% reduction in viral titer.

### Example 4

A stability study was conducted. A BENZ solution was stored for 14 days at 54° C. At Day 0, the samples contained an average of 20.26% BENZ. At Day 14, the samples contained an average of 19.97% BENZ. In this regard, nearly all chlorinated liquids degrade exceptionally quickly, but the BENZ did not.

The present disclosure has been described with reference to exemplary embodiments. Modifications and alterations will occur to others upon reading and understanding the preceding detailed description. It is intended that the present disclosure be construed as including all such modifications and alterations insofar as they come within the scope of the appended claims or the equivalents thereof.

1. A method for treating a patient with a composition, wherein the patient is suffering from a skin condition or is at risk of developing a skin condition, the method comprising the steps of:
determining a dosage of a composition comprising a halo active aromatic sulfonamide compound; and
administering the dosage of the composition to the patient;
wherein the composition comprises a halo active aromatic sulfonamide compound of Formula (I): 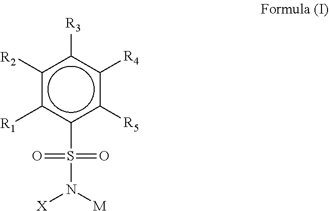
wherein R₁, R₂, R₃, R₄, and R₅ are independently selected from hydrogen, COOR′, CON(R″)₂, alkoxy, CN, NO₂, SO₃R″, halogen, substituted or unsubstituted phenyl, sulfonamide, halosulfonamide, N(R″)₂, substituted or unsubstituted C₁-C₁₂ alkyl, and substituted or unsubstituted aromatic;
R′ is hydrogen, an alkali metal, an alkaline earth metal, substituted C₁-C₁₂ alkyl, or unsubstituted C₁-C₁₂ alkyl; and
R″ is hydrogen or substituted or unsubstituted C₁-C₁₂ alkyl, where the two R″ groups in CON(R″)₂ and N(R″)₂ may be independently selected;
X is halogen; and
M is an alkali or alkaline earth metal.

2. The method of claim 1, wherein the skin condition is an infection.

3. The method of claim 1, wherein the composition further comprises at least one of: a buffering agent; a surfactant; and a solvent.

4. The method of claim 1, wherein the composition comprises from about 0.0001 wt % to 100 wt % of the halo active aromatic sulfonamide compound.

5. The method of claim 1, wherein the patient is a human or an animal.

6. The method of claim 1, wherein the skin condition includes at least one of: acne; eczema; rosacea; hives; psoriasis; athlete's foot; candidiasis; shingles; chicken pox; cold sores.

7. The method of claim 1, wherein the composition is in the form of an aerosol, a cream, an emulsion, a gel, a lotion, an ointment, a paste, a powder, a soap, a spray, a suspension, or a tape.

8. A method for treating a patient with a composition, wherein the patient is suffering from a skin condition or is at risk of developing a skin condition, the method comprising the steps of:
determining a dosage of a composition comprising a halo active aromatic sulfonamide compound;
providing an amount of the composition to a delivery device; and
administering an amount of the composition to the patient using the delivery device;
wherein the composition comprises a halo active aromatic sulfonamide compound of Formula (I): 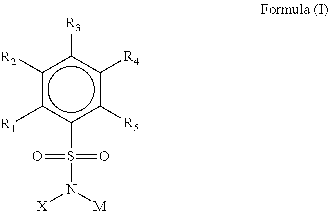
wherein R₁, R₂, R₃, R₄, and R₅ are independently selected from hydrogen, COOR′, CON(R″)₂, alkoxy, CN, NO₂, SO₃R″, halogen, substituted or unsubstituted phenyl, sulfonamide, halosulfonamide, N(R″)₂, substituted or unsubstituted C₁-C₁₂ alkyl, and substituted or unsubstituted aromatic;
R′ is hydrogen, an alkali metal, an alkaline earth metal, substituted C₁-C₁₂ alkyl, or unsubstituted C₁-C₁₂ alkyl; and
R″ is hydrogen or substituted or unsubstituted C₁-C₁₂ alkyl, where the two R″ groups in CON(R″)₂ and N(R″)₂ may be independently selected;
X is halogen; and
M is an alkali or alkaline earth metal.

9. The method of claim 8, wherein the dosage of the composition is administered by contacting an external portion of the patient with the delivery device.

10. The method of claim 8, wherein the delivery device is at least one of surgical gloves, gauze, a wound dressing, and a skin graft.

11. A composition for external administration and effective for the treatment or prevention of a skin condition in a patient, the composition comprising:
a halo active aromatic sulfonamide compound of Formula (I): 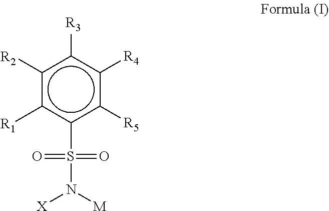
wherein R₁, R₂, R₃, R₄, and R₅ are independently selected from hydrogen, COOR′, CON(R″)₂, alkoxy, CN, NO₂, SO₃R″, halogen, substituted or unsubstituted phenyl, sulfonamide, halosulfonamide, N(R″)₂, substituted or unsubstituted C₁-C₁₂ alkyl, and substituted or unsubstituted aromatic;
R′ is hydrogen, an alkali metal, an alkaline earth metal, substituted C₁-C₁₂ alkyl, or unsubstituted C₁-C₁₂ alkyl; and
R″ is hydrogen or substituted or unsubstituted C₁-C₁₂ alkyl, where the two R″ groups in CON(R″)₂ and N(R″)₂ may be independently selected;
X is halogen; and
M is an alkali or alkaline earth metal.

12. The composition of claim 11, wherein at least one of R₁, R₂, R₃, R₄, and R₅ is not hydrogen.

13. The composition of claim 11, wherein the halo active aromatic sulfonamide compound has the structure of Formula (II):
wherein R₃ is COOR′; 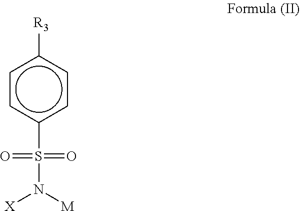
R′ is hydrogen, an alkali metal, an alkaline earth metal, substituted C₁-C₁₂ alkyl, unsubstituted C₁-C₁₂ alkyl, substituted aromatic, or unsubstituted aromatic;
X is halogen; and
M is an alkali or alkaline earth metal.

14. The composition of claim 11, wherein the halo active aromatic sulfonamide compound is N-chloro-4-carboxybenzenesulfonamide.

15. The composition of claim 11, wherein one or more of R₁, R₂, R₃, R₄, and R₅ is COOR′.

16. The composition of claim 11, further comprising water.

17. The composition of claim 11, further comprising a buffering agent.

18. The composition of claim 17, wherein the composition has a pH of about 6 to about 10.

19. The composition of claim 1, further comprising a surfactant.

20. The composition of claim 1, wherein the composition comprises from about 0.0001 wt % to about 100 wt % of the halo active aromatic sulfonamide compound.
